(19) United States
(12) Patent Application Publication
(10) Pub. No.: US 2013/0047992 A1
(43) Pub. Date: Feb. 28, 2013

(54) ENDOTRACHEAL TUBE COMPRISING CORRUGATED CUFF

(71) Applicants: Gon Hyung Kim, Chungcheongbuk-do (KR); Jin Uk Park, Seoul (KR)

(72) Inventors: Gon Hyung Kim, Chungcheongbuk-do (KR); Jin Uk Park, Seoul (KR)

(21) Appl. No.: 13/218,711

(22) Filed: Aug. 26, 2011

(51) Int. Cl. A61M 16/04

(52) U.S. Cl. 128/207.15

(57) ABSTRACT

An endotracheal tube is provided, which has a corrugated cuff and is used for purpose of establishing patent airway and artificial ventilation of a patient in fields such as anesthesiology or emergency medicine. Since the cuff of the endotracheal tube is structured to have a corrugated pattern on an outer surface when expanded, the cuff is able to not only provide original functions of preventing entry of pharyngeal content into trachea by sealing off space between the endotracheal tube and the inner wall of the trachea, and preventing gas leakage during positive pressure ventilation, but also greatly reduce incurrence of complications after use of the endotracheal tube such as ischemic damage or sore throat by minimizing contact surface between the cuff and the tracheal mucous membrane.

### BACKGROUND OF THE INVENTION

1. Field of the Invention

The present invention relates to an endotracheal tube used for purpose of establishing patent airway and artificial ventilation in fields of anesthesiology, emergency medicine, etc., and more particularly to an endotracheal tube comprising a corrugated cuff.

2. Description of the Related Art

Generally, an endotracheal tube provides an airway through which oxygen or anesthetic is directly delivered into a patient, and includes an open distal end formed on one side of a cylindrical tube of a predetermined length to be inserted into trachea and an open base portion formed on the opposite side to be connected to a gas supply.

An endotracheal tube generally has a cuff which is formed expandably on an outer portion of a cylindrical tube to thus form a sealed portion in cooperation with an inner wall of the trachea. The cuff occludes the trachea and blocks any foreign substances from entering into the trachea and lung. The cuff is particularly used to block food, external organism, or content of digestive system from being sucked into the lung. The endotracheal tube is mainly used in surgeries, but sometimes in emergency rooms or at sites of emergency. In surgery which uses general anesthesia, a patient has impaired consciousness as anesthetic including drug or gas is administered. In order to minimize vomiting of the patient in response to insertion of the endotracheal tube, muscle relaxant or paralyzant is supplied to the patient. Using laryngoscope, a practitioner pushes a tongue to a side, exerts lift with respect to the handle of the laryngoscope, to thus ensure visibility of the anatomical structure of the mouth and trachea. Larynx is the target area of the endotracheal tube, which is an opening between vocal cord and tracheal inlet. A distal end of the endotracheal tube is inserted into larynx, and the expandable cuff (i.e., balloon) is inflated with air to form air-tight sealing portion between the wall of the cuff and inner wall of the trachea. The air-tight sealing portion allows direct supply of oxygen and anesthetic gas of positive pressure into airway below the leading end and the balloon.

The cuff within the endotracheal tube plays very important roles. That is, due to the presence of the cuff of the endotracheal tube, sucking of content of the mouth or larynx is prevented, and also leakage of positive pressure ventilation gas is prevented. However, if expanded with overly high or low pressure, the cuff itself can cause various complications. For example, excessively-expanded cuff can decrease or block the flow of capillaries within the tracheal mucous membrane and subsequently result in ischemic damage, and sore throats, necrosis of tracheal mucous membrane, tracheostenosis, or in severe cases, fatal symptoms such as tracheal rupture. On the other hand, if cuff has insufficient pressure, there is high possibility of having gas leakage, pulmonary aspiration, etc. Accordingly, it is very important that the cuff is expanded with appropriate pressure. Complications such as sore throat can incur due to shape and pressure of the cuff after the treatment, and many studies have been conducted to minimize incurrence of such complications (The Journal of the Korean Society of Critical Care Medicine, v12(1): 43-48, The Korean Society of Anesthesiologists 33(5): 864-868, The Korean Society of Anesthesiologists 56:513-518). Cuffs with various shapes and functions have been continuously researched and developed so far, but none was successful in the effort to completely eliminate complications related with cuffs. Accordingly, it is necessary to develop a cuff which can provide uncompromised function, while reducing specific complications.

### SUMMARY OF THE INVENTION

Exemplary embodiments of the present inventive concept overcome the above disadvantages and other disadvantages not described above. Also, the present inventive concept is not required to overcome the disadvantages described above, and an exemplary embodiment of the present inventive concept may not overcome any of the problems described above.

In one embodiment, a cuff structure is provided, which retains the original function of the cuff, i.e., the function of preventing entry of content of pharyngeal portion into trachea by sealing between endotracheal tube and an inner wall of the trachea, and preventing gas leakage during positive pressure ventilation, and also reduces incurrence of complications such as ischemic damage or sore throat after use of endotracheal tube by minimizing a contact surface between the cuff and the tracheal mucous membrane.

In another embodiment, a cuff is provided, which is in corrugated form when expanded, to thereby minimize a contact surface with the tracheal mucous membrane, while maintaining the original function of the cuff.

In yet another embodiment, an endotracheal tube comprising a corrugated cuff is provided.

Accordingly, in one embodiment, an endotracheal tube comprising a corrugated cuff and for being inserted into trachea is provided, which may include a hollow tubular body comprising a distal end to be inserted into the trachea and a basal end portion formed opposite to the distal end, the cuff which is expandable and mounted on an outer surface of the hollow tubular body adjacent to the distal end of the hollow tubular body, and a duct connected to the cuff to introduce air to inflate the cuff, wherein the cuff has a corrugated portion when expanded.

Further, the corrugated portion on the outer surface of the expanded cuff may include a furrow and a ridge.

Further, the furrow of the cuff may be made from a material which has a low expansibility when expanded, while the ridge may be made from a material which has a high expansibility when expanded.

Further, the endotracheal tube may include a pilot balloon and an expansion valve connected to a distal end of the cuff opposite to the cuff.

Further, part of the duct may be sunken into an outer surface of the hollow tubular body.

Accordingly, since the cuff of the endotracheal tube has a corrugated shape on an outer surface when expanded, the cuff prevents entry of pharyngeal content into trachea, prevents gas leakage during positive pressure ventilation, and also prevents incurrence of complication after use of the endotracheal tube such as ischemic damage or sore throat by minimizing contact surface between the cuff and the tracheal mucous membrane.

### BRIEF DESCRIPTION OF THE DRAWINGS

The above and/or other aspects of the present inventive concept will be more apparent by describing certain exemplary embodiments of the present inventive concept with reference to the accompanying drawings, in which:

FIG. 1 is a perspective view of a conventional endotracheal tube;

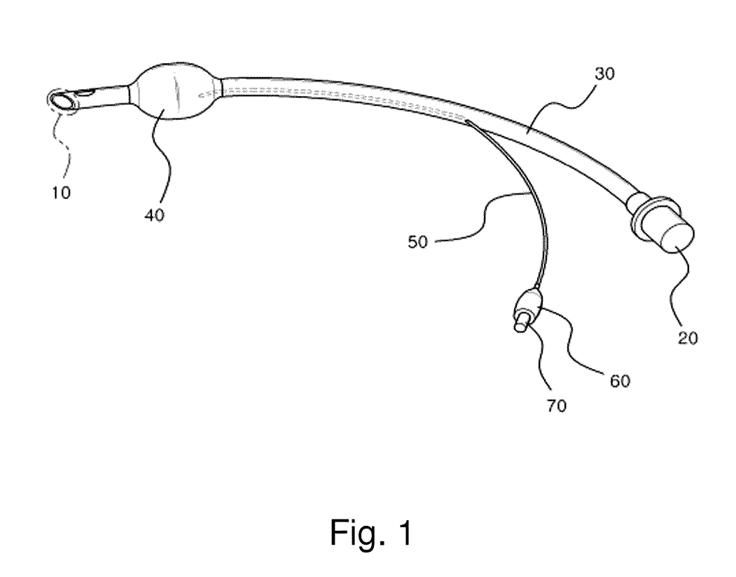

FIG. 2 is a view illustrating operational status of a low-volume, high-pressure cuff attached to a conventional endotracheal tube;

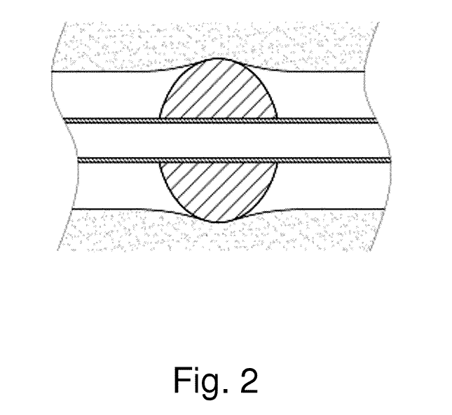

FIG. 3 is a view illustrating operational status of a high-volume, low-pressure cuff attached to a conventional endotracheal tube;

FIG. 4 is a perspective view of an endotracheal tube comprising a corrugated cuff according to an embodiment of the present invention;

FIG. 5 is a view illustrating operational status of a cuff adapted for use in an endotracheal tube comprising a corrugated cuff according to an embodiment of the present invention;

FIG. 6 is a partially-enlarged view illustrating operational status of a cuff adapted for use in an endotracheal tube comprising a corrugated cuff according to an embodiment of the present invention; and

FIG. 7 shows photographs to compare the cuff according to an embodiment of the present invention with the conventional cuff.

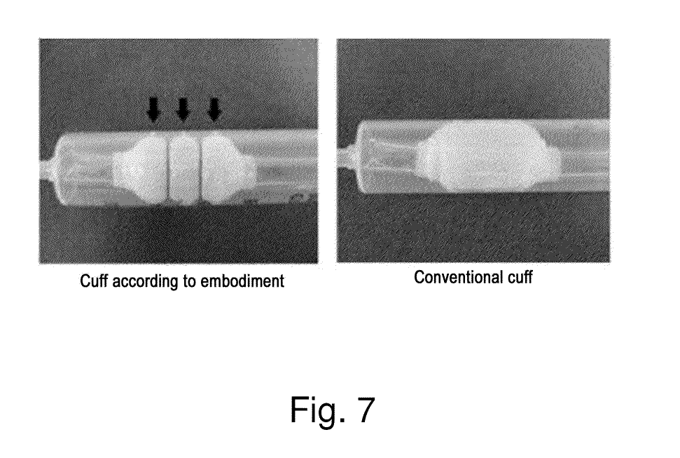

### DETAILED DESCRIPTION OF EXEMPLARY EMBODIMENTS

Certain exemplary embodiments of the present inventive concept will now be described in greater detail with reference to the accompanying drawings.

In the following description, same drawing reference numerals are used for the same elements even in different drawings. The matters defined in the description, such as detailed construction and elements, are provided to assist in a comprehensive understanding of the present inventive concept. Accordingly, it is apparent that the exemplary embodiments of the present inventive concept can be carried out without those specifically defined matters. Also, well-known functions or constructions are not described in detail since they would obscure the invention with unnecessary detail.

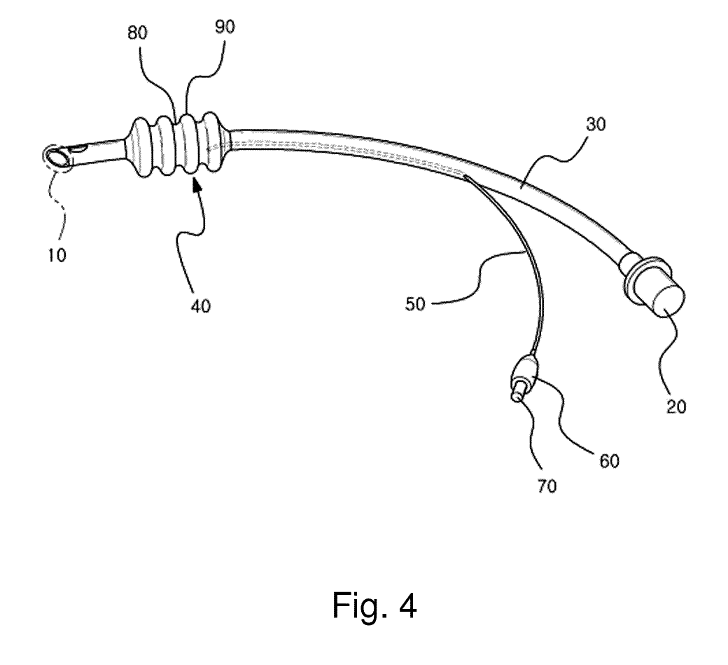

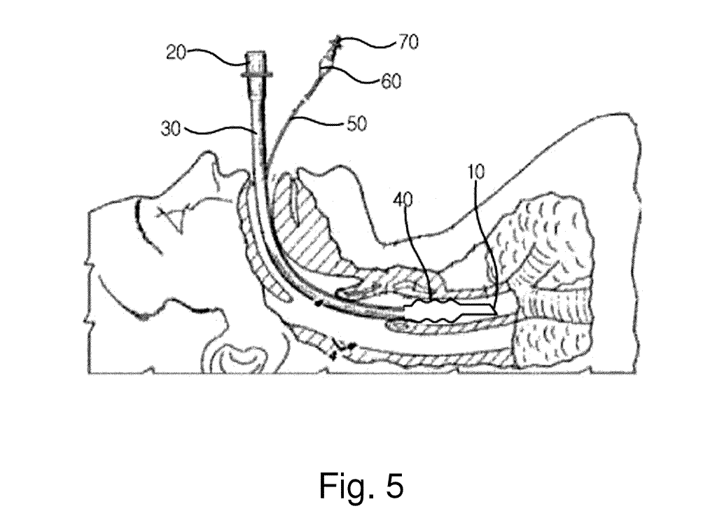

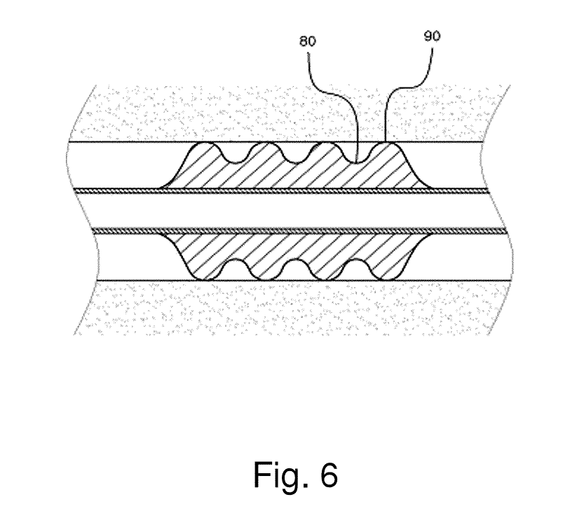

FIG. 4 is a perspective view of an endotracheal tube comprising a corrugated cuff according to an embodiment of the present invention, FIG. 5 is a view illustrating operational status of a cuff adapted for use in an endotracheal tube comprising a corrugated cuff according to an embodiment of the present invention, and FIG. 6 is a partially-enlarged view illustrating operational status of a cuff adapted for use in an endotracheal tube comprising a corrugated cuff according to an embodiment of the present invention.

The endotracheal tube having a corrugated cuff according to an embodiment may include a hollow tubular body 30 of a predetermined length provided to establish patent airway and artificial ventilation of a patient, a distal end 10 formed on one end of the hollow tubular body 30 to be inserted into trachea, a basal end portion 20 formed on an opposite side of the hollow tubular body 30, a cuff 40 mounted on the hollow tubular body 30 adjacent to the distal end 10 and expandable on an outer surface, and a duct 50 connected at one end to the cuff 40 and connected at the other end to outside.

Since the hollow tubular body 30, the basal end portion 20 and the distal end 10 are similar to those of the conventional art, no further explanation will be provided herein.

According to an aspect of the embodiment, the cuff 40, which is normally in contracted form, expands in response to introducing of air through the duct 50. As the cuff 40 expands, the cuff 40 is brought into tight contact with the inner wall of the mucous membrane of the airway, to thus block any external materials from entering into the airway.

The cuff 40 with the above-explained function may preferably be made from a specific material, such as medical polymer material which is not harmful to human body.

To be specific, the polymer material to form the cuff 40 may be one selected from polyethylene terephthalate (PETP), low density polyethylene (LDPE), polyvinyl chloride, polyurethane (PU), polyester, and polyamide.

In one embodiment, the cuff 40 may have a corrugated portion on an outer surface to allow smooth expansion and contraction, in which the corrugated portion may include furrows 80 recessed inward at predetermined intervals from each other, and ridges 90 formed in between the furrows 80 integrally and extend to outside.

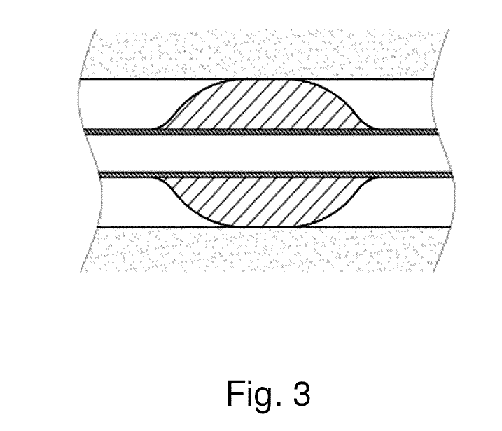

That is, since the cuff 40 in corrugated form according to an embodiment can reduce contact surface with the tracheal mucous membrane from that of a conventional cuff having high volume and low pressure as illustrated in FIG. 3, it is possible to greatly reduce risk of having ischemic damages.

To be specific, since the cuff 40 is formed into a corrugated form, when expanded, the cuff 40 is able to not only minimize the contact surface with the mucous membrane of the inner wall of the trachea, but also increase adhesion to the mucous membrane of the inner wall of the trachea and sealing of the interior of the trachea.

Furthermore, since the contact surface with the mucous membrane of the inner wall of the trachea is minimized, incurrence of complications such as ischemic damages or sore throats can be greatly reduced after use of the endotracheal tube.

Herein, while the number of the furrows 80 and ridges 90 of the cuff 40 may vary depending on the use and purpose thereof, in one embodiment, one to ten furrows 80 and ridges 90 may be formed by way of example.

That is, the number and material of the furrows 80 and ridges 90 of the cuff 40 may vary depending on the next size of the patient's larynx, or age or condition of the patient.

The furrows 80 of the cuff 40 may be made from a material which has a low expansibility when expanded, and the ridges 90 may be made from a material which has a high expansibility when expanded. However, this is written only for illustrative purpose, and accordingly, the furrows 80 and the ridges 90 may be formed from various materials in which the physical properties are appropriately adjusted. By way of example, the expansibility may be adjusted by regulating molecular weight of biomedical polymer material, cross link property, degree of cross linking, or types of side chain.

The duct 50 may have a predetermined length and may be hollow. One side of the duct 50 may be connected to the cuff 40, and the other side thereof may be placed outside the duct 50.

An expansion valve 70 may be formed on the externally-placed end of the duct 50 to maintain expansion air pressure of the cuff 40 and a pilot balloon 60, which expands in response to resistance exerted from the inner wall of the trachea.

Part of the duct 50 may be sunken into an outer surface of the hollow tubular body 30.

An embodiment will be explained in greater detail below with reference to examples.

### EXAMPLE

Applying a test method of the recent study regarding evaluation of endotracheal tube by Korean Society of Anesthesiologists (Korean J Anesthesiol, 2009, 56(5): 513-518), a disposable syringe was placed in the tracheas of a person and an animal, and endotracheal tube was inserted in the needle tube to inflate the cuff. The contact surfaces were then compared with each other.

In one embodiment, yarn was threaded on two parts of the conventional cuff to prevent inflation beyond a predetermined degree. As a result, ridges are formed, subsequently forming furrows on three portions.

The corrugated furrows had approximately 1 mm of contact surface (i.e., when calculated by length in horizontal cross-section). That is, total 3 mm of horizontal cross-section length of the contact surfaces was measured from the three furrows. Conversely, the conventional cuff had a contact surface which is 1.5 cm in horizontal cross-section length, which is five times greater than the contact surface of the cuff of the embodiment. Both the cuff of the embodiment and the conventional cuff effectively blocked airflow.

While the endotracheal tube having corrugated cuff according to the embodiments have been explained so far based on a specific shape and direction, various modifications and variations of the scope of the invention may be made by those skilled in the art within the scope of rights as protected by the invention.

The foregoing exemplary embodiments and advantages are merely exemplary and are not to be construed as limiting the present invention. The present teaching can be readily applied to other types of apparatuses. Also, the description of the exemplary embodiments of the present inventive concept is intended to be illustrative, and not to limit the scope of the claims, and many alternatives, modifications, and variations will be apparent to those skilled in the art.

What is claimed is:

1. An endotracheal tube comprising a corrugated cuff and for being inserted into trachea, comprising:
a hollow tubular body comprising a distal end to be inserted into the trachea and a basal end portion formed opposite to the distal end;
the cuff which is expandable and mounted on an outer surface of the hollow tubular body adjacent to the distal end of the hollow tubular body; and
a duct connected to the cuff to introduce air to inflate the cuff, wherein the cuff has a corrugated portion when expanded.

2. The endotracheal tube of claim 1, wherein the corrugated portion on the outer surface of the expanded cuff comprises a furrow and a ridge.

3. The endotracheal tube of claim 1, comprising a pilot balloon and an expansion valve mounted on a distal end of the cuff opposite to the cuff.

4. The endotracheal tube of claim 1, wherein part of the duct is sunken into an outer surface of the hollow tubular body.
